(19) United States
(12) Patent Application Publication
(10) Pub. No.: US 2008/0171906 A1
(43) Pub. Date: Jul. 17, 2008

(54) TISSUE PERFORMANCE VIA HYDROLYSIS AND CROSS-LINKING

(76) Inventors: Frank J.L. Everaerts, Maastricht (NL); Mark W. Torrianni, San Juan Capistrano, CA (US)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 11/623,550

(22) Filed: Jan. 16, 2007

(51) Int. Cl. A61F 2/24; A61F 2/02

(52) U.S. Cl. 600/36; 623/2.42

(57) ABSTRACT

A method for making a bioprosthetic device to reduce post-implantation mineralization of the device is provided. The method comprises providing a collagen-containing material, removing cell debris from the collagen-containing material, crosslinking the material, and removing at least a portion of ester bonds from the crosslinked collagen-containing material. Ester bonds can be removed by exposing the collagen-containing material to hydrolyzing conditions or an enzyme.

### FIELD OF THE INVENTION

This invention relates to processes for making bioprosthetic devices. More specifically, this invention relates to processes of making bioprosthetic devices that are resistant to post-implantation mineralization and calcification.

### BACKGROUND OF THE INVENTION

The surgical implantation of prosthetic devices containing natural materials, i.e., bioprosthetic devices, into humans and other mammals has been carried out with increasing frequency. Such devices include, for example, heart valves, vascular grafts, urinary bladders, heart bladders, left ventricular-assist devices, and the like. They may be constructed from natural tissues, inorganic materials, synthetic polymers, or combinations thereof.

Bioprosthetic devices materials are preferred over mechanical devices because of certain clinical advantages. For example, tissue-derived prostheses generally do not require routine anticoagulation. Moreover, when tissue-derived prostheses fail, they usually exhibit a gradual deterioration which can extend over a period of months or even years. Mechanical devices, on the other hand, typically undergo catastrophic failure.

Although any prosthetic device can fail because of mineralization, such as calcification, this cause of prosthesis degeneration is especially significant in bioprosthesis. Indeed, calcification has been stated to account for 50 percent of failures of cardiac bioprosthetic valve implants in children within 4 years of implantation. In adults, this phenomenon occurs in approximately 20 percent of failures within 10 years of implantation. See, for example, Schoen et al., J. Lab. Invest., 52, 523 532 (1985). Despite the clinical importance of the problem, the pathogenesis of calcification is not completely understood. Moreover, there apparently is no effective therapy known at the present time.

Mineralization, and especially calcification, is the most frequent cause of the clinical failure of bioprosthetic heart valves fabricated from porcine aortic valves or bovine pericardium. Human aortic homograft implants have also been observed to undergo pathologic calcification involving both the valvular tissue as well as the adjacent aortic wall albeit at a slower rate than the bioprosthetic heart valves. Pathologic calcification leading to valvular failure, in such forms as stenosis or regeneration, necessitates re-implantation. Therefore, the use of bioprosthetic heart valves and homografts have been limited because such tissue is subject to calcification. In fact, pediatric patients have been found to have an accelerated rate of calcification so that the use of bioprosthetic heart valves is contraindicated for this group.

Several possible methods to decrease or prevent bioprosthetic heart valve mineralization have been described in the literature, since the problem was first identified. Generally, these methods involve treating the bioprosthetic valve with various substances prior to implantation. Among the substances reported to work are sulfated aliphatic alcohols, phosphate esters, amino diphosphonates, derivatives of carboxylic acid, and various surfactants. Nevertheless, none of these methods have proven completely successful in solving the problem of post-implantation mineralization.

Accordingly, there is a need for providing long-term calcification resistance for bioprosthetic devices in general, and bioprosthetic heart valves in particular.

### SUMMARY OF THE INVENTION

In one aspect, a method for making a bioprosthetic device to reduce post-implantation mineralization of the device is provided. The method comprises providing a collagen-containing material, removing cell debris from the collagen-containing material, crosslinking the collagen-containing material, and removing at least a portion of ester bonds from the crosslinked collagen-containing material.

The methods is suitable for manufacturing of variety of bioprosthetic devices such as, for example, heart valves and other heart components, vascular replacements or grafts, urinary tract and bladder replacements, bowel and tissue resections, tendon replacements, and the like. The collagen-containing material includes collagen-containing tissue derived from mammals, materials comprising plant or fish collagen, and collagen-containing materials manufactured in vitro.

Removing the debris from the collagen-containing material can be achieved by any suitable method known in the art. In the preferred embodiments, such method comprises contacting the collagen-containing material with a composition comprising at least one oxidizing agent, treating the collagen-containing material with a composition comprising at least one detergent, and rinsing the collagen-containing material with a buffered solution before, between or after the other steps in the process.

In the preferred embodiments, the crosslinking of the collagen-containing material is achieved by contacting the collagen-containing material with a crosslinking solution. The crosslinking solution may comprise a crosslinking agent by itself, or it may also include a spacer, a stabilizer or both. The preferred crosslinking agent is carbodiimide. The method of crosslinking may also include a step of blocking free amine groups of the collagen-containing material prior to contacting the material with the crosslinking composition.

Ester bonds that are formed between carboxyl and hydroxyl groups of the collagen-containing material can be removed by exposing the collagen-containing material to hydrolyzing conditions or enzymes. Hydrolyzing condition comprise exposing the collagen-containing material to varying temperatures or pHs.

### BRIEF DESCRIPTION OF THE DRAWINGS

FIG. 1 illustrates calcification of the processed samples.

FIG. 2a shows magnified view of Von Kossa stained profiles of the J230 group, after 8 weeks of implantation.

FIG. 2b shows magnified view of Von Kossa stained profiles of the J230-hyd group, after 8 weeks of implantation.

FIG. 2c shows magnified view of Von Kossa stained profiles of the J400 group, after 8 weeks of implantation.

FIG. 2d shows magnified view of Von Kossa stained profiles of the J400-hyd group, after 8 weeks of implantation.

FIG. 3a shows a magnified view of J230 group, after 8 weeks of implantation.

FIG. 3b shows a magnified view of J230-hyd group, after 8 weeks of implantation.

FIG. 3c shows a magnified view of J400 group, after 8 weeks of implantation.

FIG. 3d shows a magnified view of J400-hyd group, after 8 weeks of implantation.

### DETAILED DESCRIPTION OF THE INVENTION

The applicants have discovered that removing ester bonds from a crosslinked collagen-containing device has a significant effect on the calcification pattern of the device. It was noted that hydrolyzing ester bonds shifts calcification patterns from a matrix based calcification to a cell based calcification. Although not wishing to be bound by theory, it is hypothesized that ester bonds mask nucleation sites for calcification on residual cells and cell debris in the device.

Based on this finding, the Applicants disclose a method for making a bioprosthetic device with improved mineralization resistance. Removing the cells from the collagen-containing material prior to cross-linking is believed to prevent or at least minimize post-implantation mineralization of the material. Accordingly, the method comprises providing a collagen-containing material, removing cells and cell debris from the material, crosslinking the material; and removing at least a portion of the ester bonds from the crosslinked collagen-containing material.

The methods disclosed herein are applicable to a wide variety of bioprosthetic devices such as, for example, heart valves and other heart components, vascular replacements or grafts, urinary tract and bladder replacements, bowel and tissue resections, tendon replacements, and the like. The term “bioprosthetic device” means a device made in whole or in part from collagen-containing material.

The term “collagen-containing material” includes natural materials that contain collagen as part of the extracellular matrix (ECM). One source of such materials is natural tissues. The collagen-containing material for a bioprosthetic device may be derived from mammalian species such as for example, cows, pigs, horses, chickens and kangaroos. Preferably, the collagen-containing material is a bovine or a porcine tissue such as, for example, aortic root tissue, pericardium, veins, arteries, aortic valves or hide, among others. Typically, the tissue can be obtained from a slaughter house where it can be dissected to remove undesired surrounding tissue. To reduce the degradation of the tissue, it is promptly shipped on ice to a location where the treatment of the tissue can be performed. Alternatively, the device may be manufactured from plant or fish derived collagen.

The term “collagen-containing materials” also includes collagen-containing materials manufactured in vitro. The methods for preparing collagen-containing materials in vitro are well known in the art. See e.g. U.S. Patents http://patft.uspto.gov/netacgi/nph—Parser?Sect2=PTO1&Sect2=HITOFF&p=1&u=%2Fnetahtml%2FPTO%2Fsearch—bool.html&r=1&f=G&1=50&d=PALL&RefSrch=yes&Query=PN%2F4963489—h0#h0http://patft.uspto.gov/netacgi/nph—Parser?Sect2=PTO1&Sect2=HITOFF&p=1&u=%2Fnetahtml%2FPTO%2Fsearch—bool.html&r=1&f=G&1=50&d=PALL&RefSrch=yes&Query=PN%2F4963489—h2#h24,963,489 and http://patft.uspto.gov/netacgi/nph—Parser?Sect2=PTO1&Sect2=HITOFF&p=1&u=%2Fnetahtml%2FPTO%2Fsearch—bool.html&r=1&f=G&1=50&d=PALL&RefSrch=yes&Query=PN%2F5770417-h0#h0http://patft.uspto.gov/netacgi/nph—Parser?Sect2=PTO1&Sect2=HITOFF&p=1&u=%2Fnetahtml%2FPTO%2Fsearch—bool.html&r=1&f=G&1=50&d=PALL&RefSrch=yes&Query=PN%2F5770417-h2#h25,770,417. In general, such materials are fabricated by first making a scaffold from a natural or a synthetic biocompatible and biodegradable polymer. Then, the scaffold is seeded with cells that may form extracellular matrix which includes collagen.

The collagen-containing material may be washed with a buffered solution in order to stabilize the material and assist in the removal of excess blood and body fluids that may come in contact with the tissue as applicable. A non-phosphate buffered organic solution is preferred in the present method as it serves to remove phosphate from the collagen-containing material. Using phosphate salts to buffer solutions may increase the levels of phosphate, PO₄³⁻, to the point that it will bind available divalent cations such as calcium, thus creating an environment prone to precipitate calcium phosphate salts. An organic buffer is preferred as it will typically not add additional phosphate to the collagen-containing material as do other physiologic buffers known in the art, such as sodium phosphate. Certain organic buffers also provide a buffering solution without interfering with subsequent crosslinking chemistry.

Suitable buffering agents for the non-phosphate buffered organic solutions are those buffering agents which have a buffering capacity sufficient to maintain a physiologically acceptable pH, a pH range of about 6.5 to about 8.5, and do not cause deleterious effects to the implantable medical device containing natural materials. Preferably, the non-phosphate buffered organic solution includes a buffering agent in a concentration of about 10 mM to about 30 mM. Suitable buffering agents include, but are not limited to, acetate, borate, citrate, HEPES (N-2-hydroxyethylpiperazine-N′-2-ethanesulfonic acid), BES (N,N-bis[2-hydroxyethyl]-2-amino-ethanesulfonic acid), TES (N-tris[Hydrpxymethyl]methyl-2-aminoethanesulfonic acid), MOPS (morpholine propanesulphonic acid), PIPES (piperazine-N,N′-bis[2-ethane-sulfonic acid]), or MES (2-morpholino ethanesulphonic acid). The buffering agents may be diluted in biocompatible fluids such as, for example, blood, water, or saline, among others.

The buffered solution may also comprise a chelating agent that may bind divalent cations such as calcium, magnesium, zinc, and manganese. Suitable chelating agents include, but are not limited to, EDTA (ethylenediaminetetraacetic acid), EGTA (ethylenebis(oxyethylenenitrilo)tetraacetic acid), ethylenebis(oxyethylenenitrilo)tetraacetic acid, citric acid, or salts thereof, and sodium citrate. If the chelating agents are used, they are preferably removed from the collagen-containing material prior to the step of crosslinking the collagen-containing material because the chelating agents may interfere with crosslinking agents.

The collagen-containing material is then treated to remove residual cells and cell debris. Several possible methods for removing the residual cells and debris are known in the art including physical, chemical, and biochemical methods. See e.g U.S. Pat. Nos. 5,595,571 6,121,041, and 7,078,163, which are incorporated herein by reference in their entirety. In the preferred embodiments, this step comprises contacting the collagen-containing material with a composition comprising at least one oxidizing agent, rinsing the collagen-containing material with a buffered solution, and contacting the material with a composition comprising at least one detergent.

Examples of oxidizing agents include, but are not limited to, sodium hypochlorite, sodium bromate, sodium hydroxide, sodium iodate, sodium periodate, performic acid, periodic acid, potassium dichromate, potassium permanganate, chloramine T, peracetic acid, and combinations thereof. More preferably, the oxidizing agent is selected from the group of sodium hypochlorite, performic acid, periodic acid, peracetic acid, and combinations thereof. The oxidizing agent is preferably in the composition in an amount of about 2 mM to about 20 mM, and more preferably, about 5 mM to about 10 mM. The composition may also include a buffered solution, a chelating agent or both.

The collagen-containing material is then treated with a composition comprising at least one detergent. In some embodiments, the composition may contain at least one ionic detergent and at least one non-ionic detergent simultaneously. The composition may comprise at least one zwitterionic detergent instead of at least one ionic detergent and at least one non-ionic detergent. In other embodiments, the collagen-containing material may be treated with a composition including at least one ionic detergent and a composition including at least one non-ionic detergent successively. Preferably, the collagen containing material is first treated with a composition including at least one ionic detergent. Then the collagen-containing material is treated with a composition including at least one non-ionic detergent. Suitable ionic detergents include, but are not limited to, sodium dodecyl sulfate (SDS), sodium caprylate, sodium deoxycholate, and sodium 1-decane sulfonate. The non-ionic detergents may include, but are not limited to, NP-40, Triton X-100, Tween series, and octylglucoside. The zwitterionic detergents may include, but are not limited to, a 3-(Dodecyldimethylammonio)propanesulfonate inner salt or a 3-(N,N-Dimethylmyristylammonio)propanesulfonate.

The detergent concentration in the composition may range between about 0.5% and 2.5% (weight by volume for solids or volume to volume for liquids), and more preferably between about 0.5% and 1.5%. The composition may also include a buffered solution, a chelating agent or both. The composition of this step may also optionally contain a reducing agent such as DTT (dithiothreotol) (or similar such agents) in a range of 10 mM to about 200 mM. Examples of other suitable reducing agents include, for example, 2-mercaptoethylamine and DTE (dithioerythritol).

In the preferred embodiment, the collagen-containing material may be rinsed with a buffered solution between contacting the collagen-containing material with a different composition. Buffered solutions suitable for use in this step are the same as the buffered solutions used for stabilizing the collagen-containing material as described in detail above.

In some embodiment, a number of biological components maybe added to the collagen-containing material following removal of residual cells and cell debris from the material. These components may bind to the collagen-containing material during the step of crosslinking the device. Such substances may include, for example, proteins, glycosaminoglycans (GAGs), and other bioactive substances.

Examples of proteins that may be added to the device include, but are not limited to, collagen, fibronectin, thrombin, Bone Morphogenetic Proteins (BMPs), Vascular Endothelial Growth Factors (VEGFs); Connective Tissue Growth Factors (CTGFs); Transforming Growth Factor betas (TGF-βs); Platelet Derived Growth Factors (PDGFs); Fibroblast growth factor (FGF) and combination thereof. Enzymes such as, for example, collagenase, gelatinase, serine proteases may also be used.

Suitable GAGs include, but are not limited to, chondroitin sulphate; dermatan sulphate; keratan sulphate; heparan sulphate; heparin; hyaluronan and combination thereof. Other bioactive substances may include without limitations analgesics; anti-inflammatory agents; anti-apoptotic agents; steroidal anti-inflammatory drugs such as corticosteroids; non-steroidal anti-inflammatory drugs such as salicylates; COX-2 inhibitors; opiates; morphinomimetics; and combination thereof.

Crosslinking collagen-containing material can be achieved by several methods known in the art. See e.g., U.S. Pat. Nos. 5,447,536, 5,733,339 and 7,053,051, incorporated herein by reference in their entirety. In some embodiments, the collagen-containing material may be crosslinked by contacting the material with a crosslinking solution comprising a crosslinking agent. The crosslinking agent activates the free carboxyl groups of the collagen-containing material. Reaction between a carboxyl group and the crosslinking agent yields the reactive intermediate O-acylisourea which can then react with amine groups to form amine crosslinks or react with hydroxyl groups to form ester bonds, as will be described in detail below.

Suitable crosslinking agents include, but are not limited to, a carbodiimides, an azide, 1,1′-carbonyldiimidazole, N,N′-disuccinimidyl carbonate, 2-ethoxy-1-ethoxycarbonyl-1,2-dihydroquinoline, 1,2-benzisoxazol-3-yl-diphenyl phosphate, and N-ethyl-5-phenylisoxazolium-s′-sulfonate. Preferably, the free carboxyl groups are activated by contacting them with a carbodiimide that is at least partially soluble in water. Suitable water-soluble carbodiimide include, but are not limited to, 1-ethyl-3-(3-dimethylaminopropyl) carbodiimide.HCl (EDC), cyanamide and N,N′-dicyclohexylcarbodiimide (DCC), N,N′-diisopropylcarbodiimide (DIC), and 1-cyclohexyl-3-(2-morpholinoethyl) carbodiimide metho-p-toluenesulfonate (CMC).

The crosslinking solution may also include a stabilizer, a spacer, or both. The stabilizer is used to prevent carboxyl group activated by the crosslinking agent from rearranging from O-acylisourea groups to less reactive N-acylurea groups. The addition of N-hydroxysuccinimide (NHS) is known to decrease this tendency for rearrangement. Other stabilizing agents, such as N-hydroxybenzotriazole (HOBt), N-hydroxy-5-norbornene-endo-2,3-dicarboximide (HONB), 4-dimethylaminopyridine (DMAP), and the sulfo-derivative of N-hydroxysuccinimide, are also capable of accomplishing this. Mixtures of such stabilizing agents can also be used.

The introduction of spacers may result in a more flexible device. In the preferred embodiment, a diamine spacer is employed, although other spacers such as diepoxides and diesters may also be used. Preferably, the spacer is hydrophilic. Suitable hydrophilic diamine spacers include, but are not limited to, the diamine derivatives of polyethyleneglycol and polypropyleneglycol oligomers and polymers, and polyethylene-polypropyleneglycol copolymers, such as for example O,O′-bis(3-aminopropyl)diethyleneglycol, O,O′-bis(2-aminopropyl)polypropyleneglycol, and O,O′-bis(2-aminopropyl)polyethyleneglycol. Furthermore, aliphatic diamines of two to eight carbon atoms in length are suitable spacers. This includes compounds with substitutions in the carbon chain, such as, for example, 1,4-diaminobutane, 1,6-diaminohexane, and 1,5-diamino-2-methylpentane. These spacers are available from various sources such as, for example, Aldrich Chemical Co., and Huntsman under the trade designation “JEFFAMINE.”

The step of contacting the collagen-containing material with a crosslinking solution comprising a crosslinking agent may be carried out in an aqueous solution, and more preferably, in a buffered aqueous solution having a pH of between about 4 and 9, and more preferably between about 5 and 6. The temperature of this reaction should be below that at which the collagen is denatured. Thus, although increased temperatures do increase reaction rates, the reaction is preferably performed at room temperature, i.e., 20 to 25° C., and more preferably at 21° C.

In some embodiments, free amine groups of the collagen-containing material can be blocked by various blocking agents to improve biocompatibility of the bioprosthetic device. This step is preferably carried out in an aqueous solution, and more preferably in a buffered aqueous solution having a pH between about 6 and 7. The temperature of the reaction is between about 20 and 25° C., and more preferably about 21° C.

Suitable blocking agents include, but are not limited to, N-hydroxy succinimide esters (NHS), such as acetic acid N-hydroxysuccinimide ester, sulfo-NHS-acetate, and propionic acid N-hydroxysuccinimide ester; p-nitrophenyl esters such as p-nitrophenyl formate, p-nitrophenyl acetate, and p-nitrophenyl butyrate; 1-acetylimidazole; and citraconic anhydride (reversible blocker). Additionally, the blocking agent may be selected from aldehydes such as, for example, methanal, ethanal propional, propanal, butanal, and hexanal (caproaldehyde). Epoxides such as, for example, iso-propylglycidylether and n-butylglycidylether or sulphonyl or sulphonic acid derivatives such as 2,4,6-trinitrobenzenesulfonic acid can also be employed.

During the step of contacting the collagen-containing material with a crosslinking solution, amide bonds are formed between activated carboxyl groups and amine groups. In addition to primary amide bonds, ester bonds are formed between amino acid residues containing a terminal hydroxyl group (serine, hydroxyproline, and hydroxylysine) and activated carboxyl groups of aspartic and glutamic acids. It was found that hydrolyzing the ester bonds notably changes the calcification pattern of the collagen-containing material from a matrix based calcification to the more desirable cell based calcification.

Accordingly, it is desirable to remove at least a portion of the ester bonds from the crosslinked collagen-containing material. Removal of the ester bonds may be achieved by exposing the crosslinked collagen-containing material to ester bond hydrolyzing conditions or enzymes capable of cleaving ester bonds. Preferably, the collagen-containing material is exposed to the hydrolyzing conditions for between 2 hours and 72 hours, and more preferably between about 12 and 48 hours. In some embodiments, increasing the treatment temperature may reduce exposure time. Although the temperature may be safely increased above room temperature, it should stay should be well below the temperature at which collagen may be denatured. Alternatively, the devices made from collagen-containing material may packaged and shipped under hydrolyzing conditions.

In some embodiments, the hydrolyzing factor may be temperature. In certain embodiments, the crosslinked collagen-containing material may be exposed to an initial temperature between about 2° C. and 60° C., more preferably between about 10° C. and 50° C., and even more preferably between about 18° C. and 40° C. Typically, the reaction is carried out at 37° C.

In other embodiments, the hydrolyzing condition may be based on pH. Accordingly, in certain embodiments, the crosslinked collagen-containing material may be exposed to an initial pH of between 2 and 11. The crosslinked collagen-containing material may also be treated with an acidic or basic buffered solution having a ph between 2 and 11 to hydrolyze the ester bonds. Examples of reagents used for acidic hydrolysis include, but are not limited to, hydrochloric acid, ferroacidic acid, acetic acid, phosphoric acid, and combinations thereof. Examples of reagents used for basic hydrolysis include, but are not limited to, alkali metal (e.g., sodium and potassium) phosphates, sodium borate, sodium carbonate, sodium hydrogen carbonate, and combinations thereof. Preferably, the osmolality of the hydrolyzing composition (e.g., acidic or basic buffered solution) is controlled to prevent the material from drying out, swelling, shrinking, etc. This can be done with a salt, for example.

Enzymes can also be used to remove zero-length ester crosslinks. Although suitable enzymes can include hydrolazes for hydrolyzing the ester bonds, other enzymes can also be used that do not necessarily involve hydrolysis. Examples include, but are not limited to, esterases, lipases, and the like.

In the preferred embodiments, at least a portion of the ester bonds is removed by exposing the collagen-containing material to a mildly alkaline solution. More specifically, the mildly alkaline solution is a borate buffered saline solution with a pH between about 9.5 and 10.5, and more preferably at 10. The collagen-containing material should be exposed to these conditions for approximately 12 to 24 hours.

The invention will be further described with reference to the following detailed examples. These examples are offered to further illustrate the various specific and illustrative embodiments and techniques. It should be understood, however, that many variations and modifications may be made while remaining within the scope of the present invention.

### EXPERIMENTAL EXAMPLES

Materials:

All chemicals used were obtained via Sigma Aldrich (the Netherlands) and were of ACS grade. N′-(3-dimethylaminopropyl)-N-ethylcarbodiimide (EDC) and propional were stored at 4° C. Jeffamines™ with molecular weights of 230 and 400 were also obtained. For purposes of this study the Jeffamines™ are referred to as J230 and J400. Fresh porcine aortic valves were obtained from slaughterhouses in the USA (obtained via Medtronic Santa Ana, Calif., USA), rinsed free of blood and extraneous tissue debris with 0.9% NaCl (saline). The valves were trimmed to remove excess myocardium and adventitial tissue. After cleaning the valves were again rinsed in saline solution. Subsequently, the valves were transferred to containers filled with 2-(morpholino)ethane sulphonic acid, MES buffer (0.05M, pH6.5) and stored overnight at 4° C.

Methods:

Crosslinking Method:

Table 1 shows how tissue valves were processed for the various experimental groups used in this study. Chemical processing details of the matrices are listed below

TABLE 1

| | Treatment | Sample | |
|---|---|---|---|
| Group | group | number | Treatment process |
| A | J230 | N = 5 | Tissue amine groups blocked with |
| | | | propional and EDC/NHS activated |
| | | | carboxyl groups were joined via |
| | | | J230. Samples were stored at pH |
| | | | 7.4 in HEPES buffered saline |
| B | J230-hyd | N = 5 | Tissue amine groups blocked with |
| | | | propional and EDC/NHS activated |
| | | | carboxyl groups were joined via |
| | | | J230. Samples were stored in |
| | | | borate buffered saline solution |
| | | | pH10. |
| C | J400 | N = 5 | Tissue amine groups blocked with |
| | | | propional and EDC/NHS activated |
| | | | carboxyl groups were joined via |
| | | | J400. Samples were stored at pH |
| | | | 7.4 in HEPES buffered saline |
| D | J400-hyd | N = 5 | Tissue amine groups blocked with |
| | | | propional and EDC/NHS activated |
| | | | carboxyl groups were joined via |
| | | | J400. Samples were stored in |
| | | | borate buffered saline solution |
| | | | pH10. |
| E | Fresh | N = 5 | Unprocessed fresh porcine aortic |
| | tissue | | wall. |

Step 1: Blocking of the Aortic Tissue Amine Groups:

Prior to the blocking reaction, 5 randomly selected valves were transferred to a roller bottle containing MES buffer (1000 ml, 0.05 M, pH 6.5) at room temperature. After temperature equilibration, propional (0.5 M) and NaCNBH₃ (50 mM) were added. A paddle and collar was inserted and the bottle was transferred to a roller bottle system. The blocking reaction was allowed to continue for 48 h. After the 48 h blocking reaction, valves were extensively rinsed in saline solution with volume changes 3 times daily for 3 days.

Step 2: Cross-Linking of Aortic Tissue:

At the end of the rinse cycle the roller bottles were filled with MES buffer (350 ml, 0.25M, pH 5.0) containing either J230 (0.06M) or J400 (0.06M). After a 3 hours incubation time, a concentrated solution of NHS (350 ml, 0.45 M) and a concentrated solution of EDC (350 ml, 0.9 M) both in MES buffer (0.25M, pH 5.0) containing either J230 (0.06M) or J400 (0.06M) were added. A paddle and collar was inserted and the roller bottle was closed with a hydrophobic vent cap. The cross-linking reaction was allowed to proceed for 48 h on the roller bottle system. After completion of the cross-linking reaction the valves were extensively rinsed in saline solution with volume changes 3 times daily for 3 days.

Hydrolyzing Ester Bonds

Fully processed valves were transferred from their final rinse solutions into two different holding solutions. 5 valves per group cross-linked with J230 or J400 were stored in HEPES buffered saline solution (500 ml, 10 mM, pH=7.4) or borate buffered saline solution (500 ml. 10 mM, pH 10). Both buffers contained 0.05% NaN₃.

Tissue Assessment

In vitro Characterization (Physical/Chemical Tests):

In vitro characterization was performed by a number of physical/chemical tests in order to evaluate the overall properties of the processed tissue groups. The residual tissue amine groups were characterized with a calorimetric TNBS assay, the resistance to enzymatic degradation was characterized with a combination of collagenase and pronase, the tissue shrinkage temperature was determined with differential scanning calorimetry (DSC) and the residual carboxyl groups were determined after 5-BMF labeling. FTIR analysis (Biorad Excaliber seried, USA) was performed on lyophilized porcine aortic wall samples.

In Vivo Characterization:

Aortic wall samples of the valves were subdermally implanted in a Sprague-Dawley rat model for evaluation of the degree of calcification and inflammatory response for 8 weeks. One day before the experiment was performed, 3 valves were randomly selected from each valve group and transferred from its storage solution to sterile saline. Prior to implantation, discs (8 mm in diameter), of the post sinotubular aortic wall region, were punched free from the surrounding tissue. The discs were washed with sterile saline solution (3 times for 2 min). National Institute of Health guidelines for the care and use of laboratory animals (NIH 85-23 Rev. 1985) were followed.

Male, 21 day old rats (Sprague-Dawley, CD strain) were used. After anesthetization with a mixture of halothane, N₂O and O₂, backs were shaved and disinfected using Betadine™. A mid-line incision was made in the skin and in two subcutaneous pockets created and at each side of the spine a disc was inserted with the intimal side facing the facial covering of the muscles of the back. From the J230, J400, J230-hyd and J400-hyd, 6 samples each were randomly implanted and the skin was closed with a single suture. After 8 wks, animals were anesthetized with a mixture of halothane, N₂O and O₂ followed by cervical disc relocation. Following euthanasia the sample discs with the surrounding tissue were explanted and cut into two halves. From one half of the explants, the surrounding capsule was removed and these samples were stored in HEPES containing isopropylalcohol (IPA, 20 wt %) for further quantitative calcium analysis. The other half was immersion-fixed in GA (2%) in phosphate buffered saline (PBS, 0.1 M, pH 7.4) for 24 h at 4° C. Samples were subsequently de-hydraded in a graded series of alcohols. Thereafter samples were processed trough increasing concentrations of glycol methacrylate (GMA) and eventually embedded in pure GMA. GMA blocks were then faced followed by thin sectioning to 5 μm in thickness.

Host Response to EDC Processed Porcine Aortic Wall Samples:

Host response to implanted samples were quantitatively analysed after toluidine blue staining (TB). Two independent investigators counted macrophage (MO), Giant Cell and Lymphocytes in the cellular layer at the interface of the intimal side of the samples.

Total Calcium:

The calcium concentration was determined by atomic absorption spectroscopy (AAS; Perkin Elmer Optima 3000, Fullerton, USA). Samples retrieved after explant, were removed from the storage solution, blotted free of excess buffer and then frozen in liquid nitrogen followed by lyophilization. The dry weight of each tissue sample was recorded and samples were then hydrolyzed in aqueous hydrochloric acid (110° C., 15 ml, 6M) for 24 h. After hydrolysis Di-water (10 ml) was added to each sample. The signal intensity of calcium was determined by atomic emission spectrometry (n=5 per sample). The concentration of calcium per dry weight of tissue was calculated using a calibration curve obtained with standard solutions.

Calcium Distribution in Explanted Tissue Samples:

The distribution of calcium throughout the explanted samples was determined by using image analysis of Von Kossa stained histology sections. A TB counter stain was used to increase the visibility of the matrix background. Customized image processing software (Leica Q-Win, Rijswijk, the Netherlands) was used to distinguish calcification patterns and differentiate those from non calcified portions of the tissue matrix. The calcified area of the histology section was determined and presented as a percentage of the total tissue sample area.

Statistical Analysis

A student-T test was performed on data in order to compare if statistically significant differences between samples groups occurred. The acceptance criteria were that no statistical significant difference was found if the calculated p value was less then 0.05.

Results:

In Vitro Characterization (Physical/Chemical Tests)

Table 2 summarizes all the in-vitro testing results for the various treatment groups of this study. The percentage of free amine groups and free carboxyl groups are shown along with shrinkage temperatures, and resistance to enzymatic degradation. Corrected FTIR values measured at 1176 cm⁻¹ and 1050 cm⁻¹ represent peak heights relative to absorbance measured at 2925 cm⁻¹ of ester bonds present in the tissue matrix.

TABLE 2

| | Fixation | Shrinkage | carboxyl group | amine group | Resistance to | FTIR ratio |
|---|---|---|---|---|---|---|
| | method* | temperature | concentration | concentration | enzymatic | A₁₁₇₆/A₂₉₂₅A₁₀₅₀/ |
| | N = 6 | (C.) | (% of fresh) | (% of fresh) | digestion (%) | A₂₉₂₅ |
| A | P-J230 | 74.1 ± 0.1 | 42 ± 3 | 19 ± 2 | 67.5 ± 3.2 | 1.28–1.23 |
| B | P-J230-hyd | 71.8 ± 0.5 | 51 ± 2 | 17 ± 2 | 63.4 ± 2.2 | 1.14–1.15 |
| C | P-J400 | 75.1 ± 0.2 | 47 ± 2 | 18 ± 2 | 66.2 ± 2.6 | 1.16–1.19 |
| D | P-J400-hyd | 72.0 ± 1.1 | 56 ± 2 | 20 ± 1 | 64.7 ± 3.1 | 0.95–0.98 |
| E | Fresh | 62 ± 2.1 | 100 ± 4 | 100 ± 2 | 40 ± 5 | 0.27–0.31 |

In general an increase in shrinkage temperature (Ts) was observed, caused by cross-linking. Slight but significant increases in Ts were found in the J230 compared to J400 cross-linked samples. For both the J230 and J400 group, hydrolysis lead to a significantly decreased Ts. The carboxyl group concentration was in-line with the measured Ts. During cross-linking carboxyl groups became involved and the hydrolysis reaction resulted in liberation. More carboxyl groups participated in the cross-linking reaction in the J230 group, as compared to the J400 group.

Furthermore amine groups were blocked during the process and no significant differences were found between all groups. A significantly increased resistance to enzymatic degradation was observed on all groups caused by the cross-linking process. No different resistance to enzymatic degradation was observed between the groups processed with J230 and J400. For both groups the hydrolysis reaction caused a decrease in resistance to the enzymatic degradation.

Finally increased FTIR ratios were measured at 1176 and 1050 cm⁻¹ (indicative for the presence of esters in the matrices) after cross-linking of all sample groups. In the samples cross-linked with J230, a higher absorbance was measured compared to samples cross-linked with J400. For both groups decreased absorbance values were measured after hydrolysis.

In Vivo Characterization:

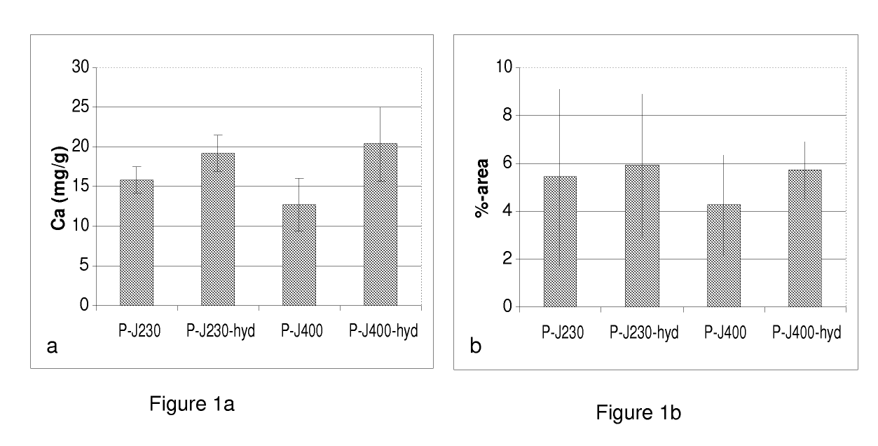

FIG. 1a represents the AAS values for explanted wall samples. The values ranged from 15.8-20.4 mg/gram of dry weight tissue. Fresh tissue data was unavailable because aortic wall samples resorbed during implant. Historically our experience with GA fixed aortic wall samples show calcium levels ranging between 60 to 80 mg/gram of dry weight tissue.

FIG. 1b is a graphical representation of the area occupied by calcific deposits in the treated tissues. The amount of calcium in μg/mg tissue is determined with AAS, while the calcification in % total area is determined using image analysis of Von Kossa stained samples. These values range from 1.2-3.7 percent of the total matrix. The area determinations appear to have the same trend with the AAS numbers for total calcium. The absolute calcification was equal in all groups.

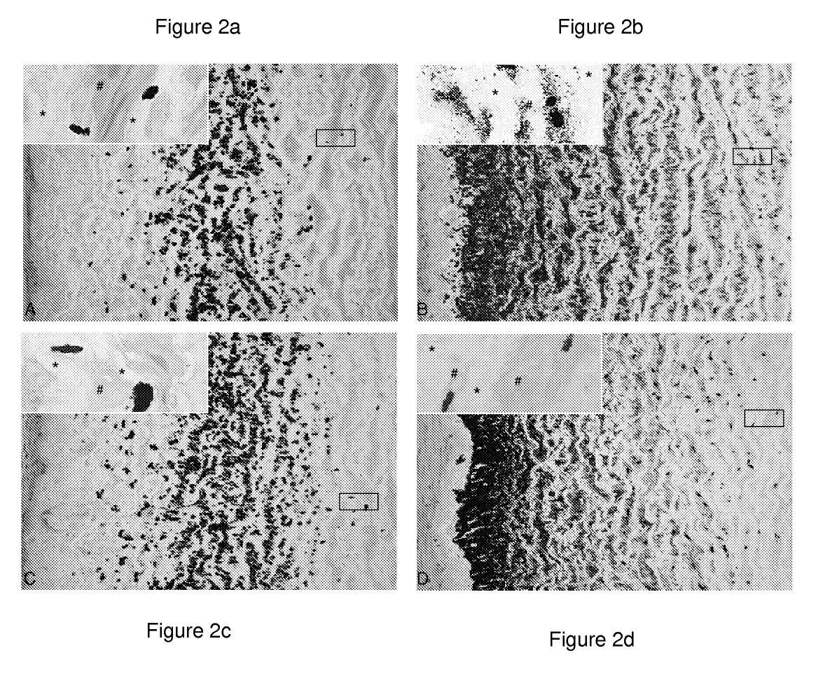

FIGS. 2a, 2b, 2c, and 2d are 200× magnifications of the Von Kossa histology slides of the of the J230, J230-hyd, J400, and J400-hyd respectively. A toluidine counterstain was used to enhance the background contrast to show the distribution of the mineral deposition within the matrix. Inset pictures are 1000× (oil immersion) magnifications of selected areas within the large panel demonstrating the orientation of mineral deposition toward either cells or extracellualr matrix. From the images it was concluded that in the absence of hydrolysis (see FIG. 2a, 2c) the calcification spots are related to the extra cellular matrix, while after completion of the hydrolysis process, calcification is related to the remaining aortic cells (FIG. 2b, 2d). Furthermore there were changes in the pattern of calcification in that without hydrolysis, calcification is induced in the inner wall tissue while after hydrolysis calcification is concentrated on the adventicial side of the sample.

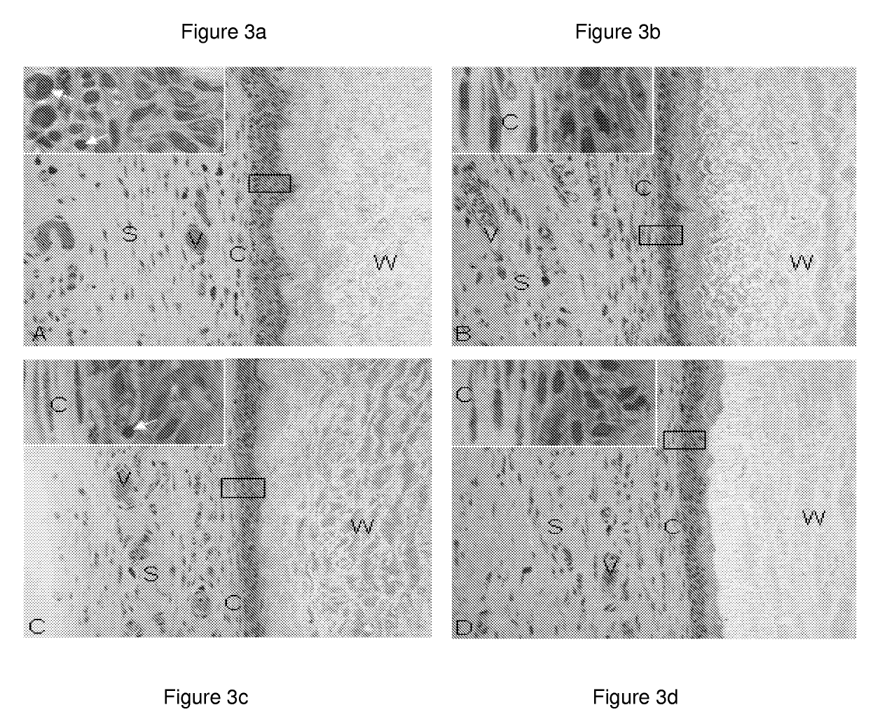

FIGS. 3a, 3b, 3c, and 3d is a panel of Toluidine Blue stained histology sections of the J230, J230-hyd, J400, and J400-hyd respectively. In each of the panels the large micrographs represent the implant and the tissue interface of the surrounding capsule. Aortic wall sections are seen at the right in each panel with the capsule on the left separated by a layer of inflammatory cells. The inserts are high magnification images of the cellular composition of the inflammatory cell layer between the implant and the host tissue. The capsule (C) and the surrounding tissue (S) are populated with small blood vessels (V). The Interface (.), between the aortic wall (W), is populated with macrophages and lymphocytes.

Histological analysis after TB staining revealed no significant differences in the foreign body reaction between all groups. The absolute number of macrophages and giant cells was equally low and a small layer of these cells was only observed at the interface of the wall tissue. Furthermore, low numbers of lymphocytes were observed at the interface of the intimal side, but no significant differences in the amounts of lymphocytes were measured. Within all groups a small capsule had been formed around the interface and some blood vessels were present in the surrounding tissue

All publications cited in the specification, both patent publications and non-patent publications, are indicative of the level of skill of those skilled in the art to which this invention pertains. All of these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A method for making a bioprosthetic device to reduce post-implantation mineralization of the device comprising:
providing a collagen-containing material;
removing cell debris from the collagen-containing material;
crosslinking the collagen-containing material; and
removing at least a portion of ester bonds from the crosslinked collagen-containing material.

2. The method of claim 1 further comprising adding biological components to collagen-containing material.

3. The method of claim 1, wherein the collagen-containing material is selected from the group consisting of porcine aortic root tissue, bovine aortic root tissue, porcine pericardium, bovine pericardium, bovine veins, porcine veins, bovine arteries, porcine arteries, porcine aortic valves, bovine aortic valves, porcine hide, and bovine hide.

4. The method of claim 1, wherein the collagen-containing material is manufactured in vitro.

5. The method of claim 1, wherein the bioprosthetic device is selected from the group consisting of heart valves and other heart components, vascular replacements or grafts, urinary tract and bladder replacements, bowel and tissue resections, and tendon replacements.

6. The method of claim 1, wherein the bioprosthetic device is a heart valve.

7. The method of claim 1, wherein the step of removing cell debris from the collagen-containing material comprises:
contacting the collagen-containing material with a composition comprising at least one oxidizing agent;
rinsing the collagen-containing material with a non-phosphate buffered solution; and
treating the collagen-containing material with a composition comprising at least one detergent.

8. The method of claim 7, wherein the step of treating the collagen-containing material with a composition comprising at least one detergent comprises treating the collagen-containing material with a composition comprising at least one ionic detergent and at least one non-ionic detergent.

9. The method of claim 7, wherein the step of treating the collagen-containing material with a composition comprising at least one detergent comprises:
treating the collagen-containing material with a composition comprising at least one ionic detergent;
treating the collagen-containing material with a composition comprising at least one non-ionic detergent;
rinsing the collagen-containing material with buffered solution between the steps of treating the collagen-containing material with compositions comprising the at least one ionic and the at least one non-ionic detergents.

10. The method of claim 1, wherein the step of crosslinking the collagen-containing material comprises contacting the collagen-containing material with a crosslinking solution.

11. The method of claim 8, wherein the step of crosslinking the collagen-containing material further comprises:
treating the collagen-containing material with an agent adapted to block amine groups of the collagen-containing material.

12. The method of claim 8, wherein the crosslinking solution comprises a crosslinking agent by itself or in combination with a stabilizer or a spacer.

13. The method of claim 12, wherein the crosslinking agent is selected from the group consisting of a carbodiimide; an azide; 1,1′-carbonyldiimidazole; N,N′-disuccinimidyl carbonate; 2-ethoxy-1-ethoxycarbonyl-1,2-dihydroquinoline, 1,2-benzisoxazol-3-yl-diphenyl phosphate; and N-ethyl-5-phenylisoxazolium-s′-sulfonate; and combinations thereof.

14. The method of claim 12, wherein the stabilizer is selected from the group consisting of N-hydroxysuccinimide (NHS); N-hydroxybenzotriazole (HOBt); N-hydroxy-5-norbornene-endo-2,3-dicarboximide (HONB); 4-dimethylaminopyridine (DMAP); sulfo-derivative of N-hydroxysuccinimide and combinations thereof.

15. The method of claim 12, wherein the spacer is a diamine spacer.

16. The method of claim 1, wherein the step of removing at least a portion of the ester bonds from the crosslinked collagen-containing material comprises exposing the crosslinked collagen-containing material to ester bond hydrolyzing conditions.

17. The method of claim 16, wherein the ester bond hydrolyzing conditions comprise exposing the crosslinked collagen-containing material to a basic buffered solution.

18. The method of claim 17, wherein the basic buffered solution is a borate buffered solution with a pH between about 9 and 11.

19. The method of claim 16, wherein the ester bond hydrolyzing conditions comprise exposing the crosslinked collagen-containing material to an acidic buffered solution.

20. The method of claim 19, wherein the acidic buffered solution has a pH between about 4 and 5.
